(19) United States
(12) Patent Application Publication
(10) Pub. No.: US 2024/0374911 A1
(43) Pub. Date: Nov. 14, 2024

(54) Methods and Apparatuses for Wearable Neuromodulation Pain Relief Device

(71) Applicant: MUONX, LLC, Minersville, PA (US)

(72) Inventors: Edward Dunleavy, Minersville, PA (US); Lori L. Dunleavy, Minersville, PA (US)

(21) Appl. No.: 18/291,968

(22) PCT Filed: Jul. 27, 2022
(86) PCT No.: PCT/US22/74189

§ 371 Date: Jan. 25, 2024

Related U.S. Application Data
(60) Provisional application No. 63/227,562, filed on Jul. 30, 2021.

(51) Int. Cl. A61N 2/00; A61N 2/02

(52) U.S. Cl. CPC A61N 2/008; A61N 2/02

(57) ABSTRACT

The present invention provides a method and apparatus for pain relief. In one embodiment, the present invention provides a passive electromagnetic apparatus to relieve pain using established electrochemical principles in conjunction with alloy composition elements and designs having electromagnetic characteristics to attract, transport and dissipate electrochemical ions in nerve cell bodies to relieve pain when applied on or near a painful area.

### CROSS-REFERENCE TO RELATED APPLICATIONS

This application claims priority to U.S. provisional application No. 63/227,562 filed on Jul. 30, 2021, incorporated herein by reference in its entirety.

### BACKGROUND OF THE INVENTION

Present art for targeted pain relief include implanted electric powered neuromodulation and topical transcutaneous electric nerve stimulation (TENS) devices, ingestible and topical pharmaceuticals, and physical or psychological therapies.

The International Neuromodulation Society defines therapeutic neuromodulation as “the alteration of nerve activity through targeted delivery of a stimulus, such as electrical stimulation or chemical agents, to specific neurological sites in the body” (International Neuromodulation Society, “How has Neuromodulation Been Developed and Used?” https://neuromodulation.com/learn-more). Neuromodulation therapies help to re-establish normal function of the nervous system. One of the most common examples of neuromodulation is the use of spinal cord stimulation (SCS) for chronic pain management. SCS consists of a very thin lead or wire that is placed in the space just outside the spinal cord known as the epidural space. The lead is attached to a small generator device that is implanted under the skin and subcutaneous layer in the back or buttock. The devices will deliver frequent, low-voltage electrical impulses to the spine, with subsequent modulation of the pain signals in transit to the brain. Those impulses often feel like a gentle tingling or buzzing which is called paresthesia on the body. There has been significant advancement in the hardware and the technology since the first model was placed, and patients report better pain control with less feeling of vibrations.

Prescription, over the counter medications, biofeedback, neuromodulation, acupuncture, physical and psychological manipulative techniques are popular interventions to provide pain relief. The American College of Physicians (ACP) and the American Academy of Family Physicians (AAFP) recommend that topical nonsteroidal anti-inflammatory drugs (NSAIDs) should be first-line treatment for acute pain from musculoskeletal injuries. Other prescription medications like antidepressants and opioids provide pain relief however other unintended consequences occur such as sleepiness, weight gain, or worse, addiction or overdose.

Neuromodulation, TENS, acupuncture/acupressure are less risky and sometimes helpful for pain relief, however these forms of therapy are expensive and require mechanical power for results. Further, other disadvantages of the current methods include: expense of tiered professional consultations for dispensing; time constraints to obtain appointments for tiered medical professions; income loss due to pain presentment; concomitant effects of medication and device trials; unknown outcomes; implants may limit future imaging diagnostics like MRI or CT scans due to photonic scattering obscuring images; and present art wearable pain relief technology require power sources to function.

Thus, there is a need in the art for a pain remediation device and method that is convenient for the patient, requires no stored energy to operate and is affixed on or near a painful area of the body appendage. The present invention meets this need.

### SUMMARY OF THE INVENTION

In one aspect, the present invention provides a therapeutic device for pain relief comprising: an electrode coupler base, and a two lead single length wire inductor, wherein the wire inductor comprises at least one integrated Helmholtz coil. In one embodiment, the electrode coupler base is made from a material selected from the group consisting of: alloys of stainless steel, iron, zinc, nickel chromium, silver sheets and combinations thereof. In one embodiment, the electrode coupler base has a shape selected from the group consisting of: arched, round, rectangle, square, keystone, flat, curve, hollow curve tube, straight tube shape and combinations thereof. In one embodiment, the electrode coupler base further comprises at least two slot openings, configured to allow attachment of the electrode coupler to the two lead wire inductor. In one embodiment, the two lead wire inductor are inserted within the at least two slot openings and completing a closed loop electromagnetic circuit, allowing the attraction of sodium ions. In one embodiment, the at least one integrated Helmholtz coil comprises at least one turn in the coil. In one embodiment, the at least one integrated Helmholtz coil is positioned anywhere on the length of the two lead wire inductor. In one embodiment, the two lead wire inductors further comprise a securing clasp positioned at each end of the two lead wire inductors and is configured to secure the electrode coupler base to the two lead wire inductors. In one embodiment, the securing clasp has a length ranging between approximately 3-5 mm. In one embodiment, the one lead of the wire inductor further comprises a sharp ninety degree left bend. In one embodiment, the electrode coupler base is shaped as an arched half round and half rectilinear with two triangular shape cutouts that are used as compression hems. In one embodiment, the two lead wire inductor is made from a material selected from the group consisting of: alloys of stainless steel, iron, zinc, nickel chromium, high carbon steel, low carbon steel, silver sheets and combinations thereof. In one embodiment, the electrode base coupler is attached to two lead wire inductor via a method selected from the group consisting of: high strength epoxy adhesives, metal alloy soldering, Bondic [TM] Ultraviolet activated glue, and combinations thereof. In one embodiment, the device is fixed to the skin of a subject by a method selected from the group consisting of: an adhesive bandage tape, an appendage fabric material, a fabric hook and loop belt material or combinations thereof.

In one aspect, the present invention provides a method of providing pain relief to a subject comprising the steps of: providing a device having an electrode coupler base, and a two lead single length wire inductor, wherein the wire inductor comprises at least one integrated Helmholtz coil; attaching the two lead wire inductor to the electrode coupler base; and affixing the device to the skin of the subject. In one embodiment, the electrode base coupler is attached to two lead wire inductor via a method selected from the group consisting of: high strength epoxy adhesives, metal alloy soldering, Bondic [TM] Ultraviolet activated glue, and combinations thereof. In one embodiment, the electrode coupler base further comprises at least two slot openings, configured to allow attachment of the electrode coupler to the two lead wire inductor. In one embodiment, the two lead wire inductor are inserted within the at least two slot openings and completing a closed loop electromagnetic circuit, allowing the attraction of sodium ions. In one embodiment, the device is fixed to the skin of a subject by a method selected from the group consisting of: an adhesive bandage tape, an appendage fabric material, a fabric hook and loop belt material or combinations thereof.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1C, depicts an exemplary wearable therapeutic pain remediation device of the present invention. FIG. 1A depicts an exploded obverse side of the exemplary wearable therapeutic pain remediation device.

FIG. 1B depicts an assembled reverse side of an exemplary wearable therapeutic pain remediation device of the present invention. FIG. 1C depicts an assembled obverse side of an exemplary wearable therapeutic pain remediation device of the present invention.

FIG. 2 depicts a computer aided design template fabrication dimensions for an exemplary wearable therapeutic pain remediation device of the present invention.

FIG. 3 depicts a cross-sectional view of an exemplary wearable therapeutic pain remediation device of the present invention.

FIG. 4, comprising FIG. 4A through FIG. 4B, depicts a side view of an exemplary wearable therapeutic pain remediation device of the present invention when affixed to the subject. FIG. 4A depicts an exemplary wearable therapeutic pain remediation device of the present invention applied to an adhesive tape bandage. FIG. 4B depicts an obverse and reverse sides of wearable therapeutic device inserted in a fabric headband or a fabric hook and loop belt.

FIG. 5, comprising FIG. 5A through FIG. 5D, depicts another exemplary wearable therapeutic pain remediation device of the present invention. FIG. 5A depicts an exploded view of an exemplary wearable therapeutic pain remediation device of the present invention. FIG. 5B depicts an assembled view of an exemplary wearable therapeutic pain remediation device of the present invention. FIG. 5C depicts an exploded view of an exemplary wearable therapeutic pain remediation device of the present invention with a skewed Helmholtz coil and securing clips. FIG. 5D depicts an assembled view of an exemplary wearable therapeutic pain remediation device of the present invention with a skewed Helmholtz coil and securing clips.

FIG. 6, comprising FIG. 6A through FIG. 6B, depicts another exemplary wearable therapeutic pain remediation device of the present invention. FIG. 6A depicts an exploded view of an exemplary wearable therapeutic pain remediation device of the present invention. FIG. 6B depicts an assembled view of an exemplary wearable therapeutic pain remediation device of the present invention.

FIG. 7, comprising FIG. 7A through FIG. 7D, depicts another exemplary wearable therapeutic pain remediation device of the present invention. FIG. 7A depicts a close up view of an obverse and reverse side of the electrode coupler base. FIG. 7B depicts a layered view of the reverse side wearable therapeutic device showing the keystone column shape formation of a square electrode coupler base and a two lead elongated trombone slide shape wire inductor wire with integrated Helmholtz coil and a ninety degree return securing post inserted under the three hemmed rectangle folds. FIG. 7C depicts an obverse side of the assembled view of an exemplary wearable therapeutic pain remediation device of the present invention. FIG. 7D depicts a reverse side of the hemmed assembly of an exemplary wearable therapeutic pain remediation device of the present invention.

FIG. 8, comprising FIG. 8A through FIG. 8C, depicts another exemplary wearable therapeutic pain remediation device of the present invention. FIG. 8A depicts an exploded view of the reverse side of the electrode coupler of arched half round and half rectilinear shape with three triangle compression hem fold over cutouts and two lead half arched return elongated trombone slide shape wire inductor with integrated Helmholtz coil. FIG. 8B depicts a layered reverse side view of the assembled device with the electrode coupler having triangle hem folds over the two lead elongated trombone slide shape wire inductor with integrated Helmholtz coil. FIG. 8C depicts an obverse side of the assembled exemplary wearable therapeutic pain remediation device of the present invention.

FIG. 9, comprising FIG. 9A through FIG. 9C, depicts another exemplary wearable therapeutic pain remediation device of the present invention. FIG. 9A depicts an exploded view of exemplary device of the present invention with the electrode coupler base having four vertical rectangle slot openings and showing a two lead elongated trombone slide shape wire inductor with integrated Helmholtz coil and forming a securing retention post and a horizontal one hundred eighty degree return compression clasp. FIG. 9B depicts an obverse side of the assembled exemplary wearable therapeutic pain remediation device of the present invention, showing one of the two leads of the elongated trombone slide shape wire inductor with integrated Helmholtz coil loop inserted through the electrode coupler and clasped to the first lead retention post. FIG. 9C depicts a cross sectional view of an electrode coupler formed with one of two dome bridge pin slot openings dappled and one of the two leads of the elongated trombone slide shape wire inductor with integrated Helmholtz coil inserted.

FIG. 10, comprising FIG. 10A through FIG. 10B, depicts another exemplary wearable therapeutic pain remediation device of the present invention. FIG. 10A depicts an exploded view of an exemplary wearable therapeutic pain remediation device of the present invention with the straight tube electrode coupler and multi-angle, two lead wire inductor with three integrated Helmholtz coils and two hook end lead clasps. FIG. 10B depicts an assembled view of an exemplary wearable therapeutic pain remediation device of the present invention.

FIG. 11, comprising FIG. 11A through FIG. 11B, depicts another exemplary wearable therapeutic pain remediation device of the present invention. FIG. 11A depicts an exploded view of an exemplary wearable therapeutic pain remediation device of the present invention with the straight tube electrode coupler and multi-angle, two lead wire inductor with five integrated Helmholtz coils and two hook end lead clasps. FIG. 11B depicts an assembled view of an exemplary wearable therapeutic pain remediation device of the present invention.

FIG. 12, comprising FIG. 12A through FIG. 12B, depicts another exemplary wearable therapeutic pain remediation device of the present invention. FIG. 12A depicts an exploded view of an exemplary wearable therapeutic pain remediation device of the present invention showing a rectangle electrode coupler with two horizontal slot openings and a circular ring shape two lead wire inductor with two hook end lead clasps and integrated ninety degree horizontally askew Helmholtz coil. FIG. 12B depicts an assembled view of an exemplary wearable therapeutic pain remediation device of the present invention.

FIG. 13, comprising FIG. 13A through FIG. 13B, depicts another exemplary wearable therapeutic pain remediation device of the present invention. FIG. 13A depicts an exploded view of an exemplary wearable therapeutic pain remediation device of the present invention with a common spring wire clasp with a slotted half round and half rectilinear electrode coupler clasp compressed to an elongated spring wire with integrated Helmholtz coil two lead wire inductor. FIG. 13B depicts a modified exemplary therapeutic pain remediation device of the present invention with protective safety enhancements including compression of clasp slots to prevent skin abrasion from underlying muscle movement and accidental pin stabbing potential with wearing such a device on skin along with adhesive sealant permitting safe use of a common coil spring wire and clasp device.

FIG. 14, comprising FIG. 14A through FIG. 14B, depicts various locations where the wearable therapeutic device embodiments may be placed for appendage pain relief. FIG. 14A depicts the back view of the subject with the various locations for the wearable therapeutic device of the present invention. FIG. 14B depicts the front view of the subject with the various locations for the wearable therapeutic device of the present invention.

FIG. 15 depicts Linus Pauling's Electronegativity Scale of elements.

FIG. 16, comprising FIG. 16A through FIG. 16C, depicts the mechanism of sodium ion gate and the effects of the exemplary device of the present invention on voltage gated sodium ion channel action potential charge mobility. FIG. 16A is a flowchart depicting the nerve cell body voltage in resting, active, and refractory states. FIG. 16B depicts the magnetic field polarity of each of the cell states. FIG. 16B depicts the magnetic repulsion effects of like magnetic fields of the exemplary device of the present invention on electronegative sodium ion mobility in the extracellular and intracellular space. The device affects electronegative sodium ion mobility by attracting sodium ions having a Pauling Unit value of 93 from nerve cell membranes, which are necessary to communicate pain signals to adjacent nerve cells, and transports the sodium ions to elements in the therapeutic device alloy composition having a Pauling Unit values equal to or greater than 1.70 Pauling Units. The device affects sodium ion mobility in three ways: first by attracting extracellular sodium ions to the device by means of Pauling's Electronegative Scale of elements using device materials having elements of high electronegative value to attract low value sodium ions outside the neuron body extracellular space; second by using a Helmholtz coil in the exemplary device to dissipate magnetic fields within the coil; and third by reinforcing a repulsion force of like negative magnetic field polarity preventing a positive magnetic polarity change in the nerve cell bodies (FIG. 16C), keeping the nerve cell bodies in a continual negative polarity resting state and preventing formation of a voltage action potential in the nerve cell body. The combination of actions, prevent signal activation and transmission to adjacent nerve cell bodies.

FIG. 17 is a flowchart depicting an exemplary method of relieving pain using the wearable therapeutic pain remediation device of the present invention.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of targeted pain relief. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Wearable Therapeutic Pain Remediation Device

The present invention provides a wearable therapeutic pain remediation device configured to provide a safe, easy to use, lightweight, flexible, and economical pain remediation and relief. In one embodiment, the wearable device is able to attach to most body appendages. In one embodiment, the wearable device may be used for pain relief of nerve disorders including but not limited to dental pain, burning mouth syndrome, cervical and lumbar spine pain, joint (hip, knee, elbow, wrist, hand and fingers, shoulder, ankle) pain, neuro-dermatitis skin nerve itch. In one embodiment, the device of the present invention can be used to prevent prodromal migraine symptoms. In one embodiment, the wearable device of the present invention may be used for internal organ disease such as intractable cancer, organ, and bone pain. In one embodiment, the wearable device of the present invention can be easily applied on different body appendages and curvatures. In one embodiment, the wearable device of the present invention allows continuous reusability and excellent corrosion resistance. In one embodiment, the wearable device of the present invention may have a compact size that is comfortable to wear and may be discretely camouflaged when worn on exposed skin. In one embodiment, the wearable device of the present invention provides electrically passive operation and eliminates batteries replacement costs and hazardous waste in the environment. In one embodiment, the wearable device of the present invention can be used with many different materials and thus allows for trial and permanent device versions as well as material substitutions for allergy sensitive individuals with minimal impact on effectiveness. In one embodiment, the wearable device of the present invention provides near instantaneous targeted pain relief.

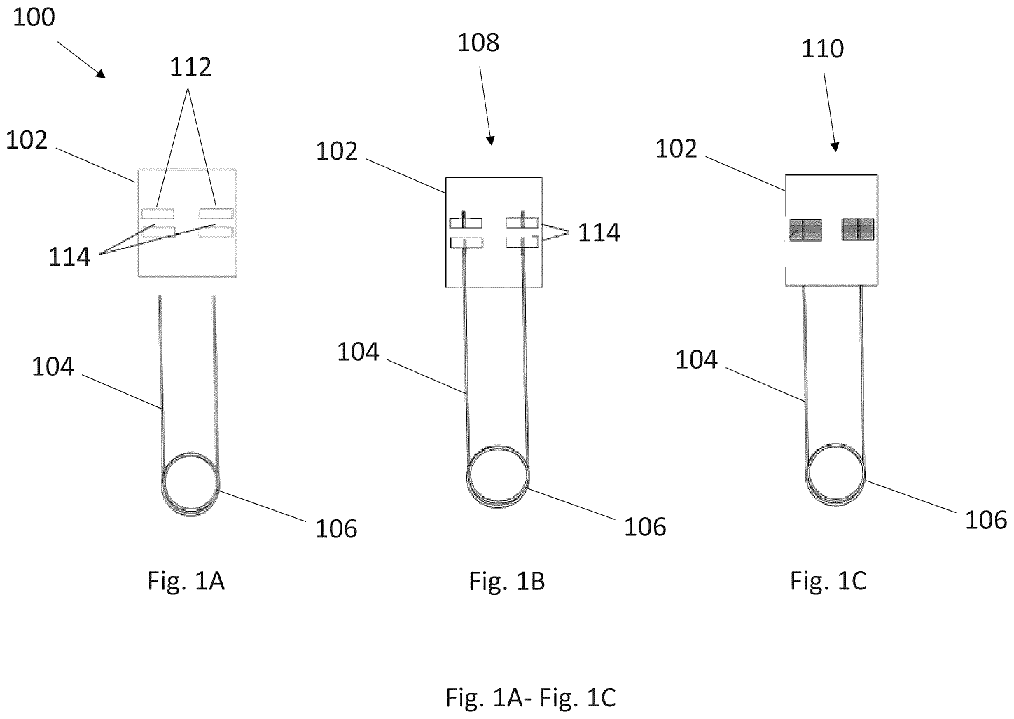

Referring now to FIG. 1A through FIG. 1C, an exemplary pain remediation device 100 is shown. Pain remediation device 100 comprises an electrode coupler base 102, two elongated wire inductors 104 and at least one integrated Helmholtz coil 106.

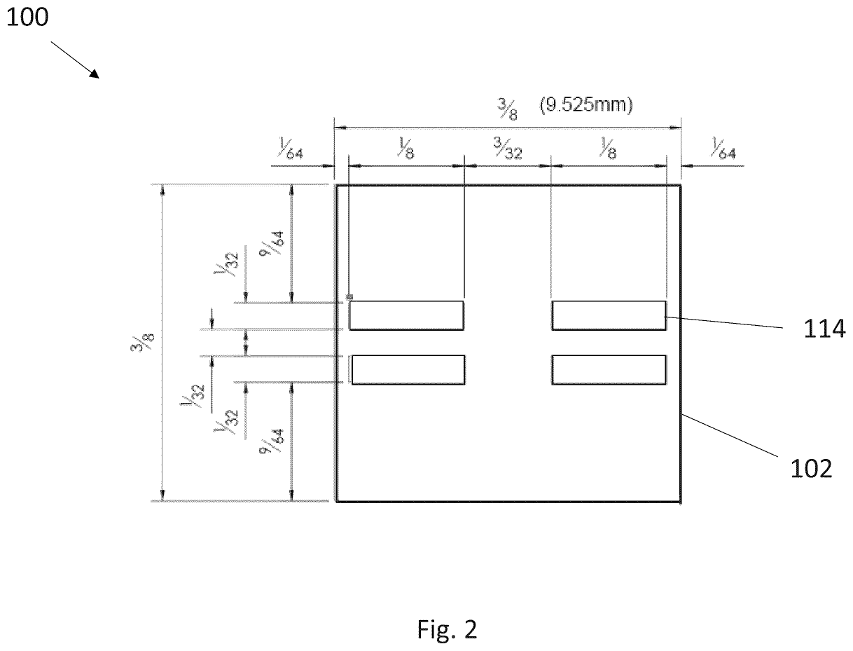

Referring now to FIG. 2, in one embodiment, electrode coupler base 102 may be made from any material known to one skilled in the art including but not limited to alloys of stainless steel, iron, zinc, nickel chromium, high carbon steel, low carbon steel, silver sheets, etc. In one embodiment, electrode coupler base 102 may have a metal sheet gauge size ranging between 30 through 18. In one embodiment, electrode coupler base 102 may have a thickness ranging between 0.318-1.219 mm. In one embodiment, electrode coupler base 102 may have an overall dimension ranging between 9.525-12.7 mm square shape. In one embodiment, electrode coupler base 102 may have an overall dimension of 12 mm in length and 9 mm in width. In one embodiment, the outer corners of electrode coupler base 102 may be beveled or rounded to avoid sharp edges against the subject's skin. In one embodiment, final shaping of the device may be achieved by flattening electrode coupler base 102 and reverting flat any unwanted curvatures using pressure blows by a steel hammer or pressure devices and alternatively using a sanding grinder or file as necessary to remove sharp edges. In one embodiment, electrode coupler base 102 may be subjected to base size cutting, edge smoothing and rounding.

In one embodiment, electrode coupler base 102 may have any shape known to one skilled in the art including but not limited to arched, round, rectangle, square, keystone, flat, curve, hollow curve tube, or straight tube shapes. In one embodiment, electrode coupler base 102 may have any shape and dimension requiring customization for various pain locations and appendage contours. For example, electrode coupler base 102 may be made from metal to allow customization to provide various bending and stretching based on pain location and appendage contour. In one embodiment, electrode coupler base 102 may be made from light weight metal alloy elements capable of bending and conforming to appendage contours. In one embodiment, electrode coupler base 102 may have compositions with the ability of attracting and conducting electromagnetic fields. In one embodiment, electrode coupler base 102 may be made of materials configured to attract sodium ions. In one embodiment, electrode coupler base 102 may be made of materials that aid in concealability. In one embodiment, electrode coupler base 102 may be made of materials that is comfort for the subject to wear. In one embodiment, electrode coupler base may be any dimension sizes to accommodate customization when required for specialized use such as in facial applications to aid in concealability.

In one embodiment, electrode coupler base 102 may be obtained and processed by any method known to one skilled in the art including but not limited to laser cutting.

Electrode coupler base 102 comprises a reverse side 108 and an obverse side 110. In one embodiment, electrode coupler base 102 comprises two columns 112, each having two slot openings 114 on reverse side 108 (FIG. 1B and FIG. 1C). In one embodiment, slot openings 114 may have any shape known to one skilled in the art including but not limited to horizontal. In one embodiment, the distance between slot openings 114 in one columns may be ranging between approximately 0.5-1 mm. In one embodiment, the distance between slot openings 114 in one row may be ranging between approximately 3-4 mm.

In one embodiment, electrode coupler base 102 may have a uniform cross section capable of allowing flexibility and repeated bending and shaping with minimal tearing or unwanted perforations to allow creation of slot openings 108. In one embodiment, slot openings 108 may have any shape known to one skilled in the art including but not limited to circular, rectangular, etc.

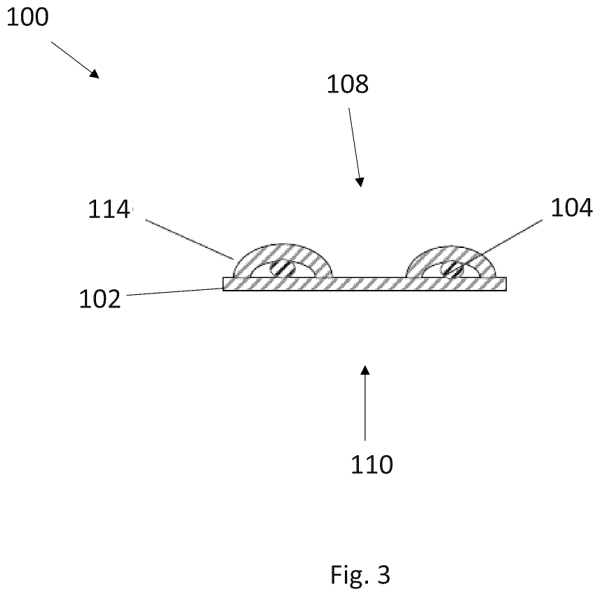

In one embodiment, slot openings 108 may be made with any method known to one skilled in the art including but not limited to laser cutting, pressure shearing machine, etc. In one embodiment, slot openings 108 may have a dome shape (FIG. 3). In one embodiment, dome shape openings may be made with any method known to one skilled in the art including but not limited to a dapple doming tool. In one embodiment, dapple doming tool is applied in the middle of each column 112, bridging the two rows of slot openings 108 to permit insertion of wire inductors 104.

In another exemplary embodiment, the dome shape slot openings 108 of the electrode coupler 102 can be formed using a dapple doming tool and applying pressure force in the middle of one of the two thin electrode coupler bridges (FIG. 1B, FIG. 3) of each column 112 by centering the electrode coupler base bridge over a 9.1 mm hole drilled into hardened metal sheet of sufficient thickness, approximately 3.125 mm to allow pressure bending of the dome bridge with the dapple doming tool without tearing or shearing the electrode coupler base 102 material and using repeated strikes by a steel hammer or other pressure bending tool or machine.

Wire inductor 104 is a single length wire inductor with two electroconductive leads at a first end and at least one integrated Helmholtz coil spring wire 106 at a second end. In one embodiment, at least one Helmholtz coil spring wire 106 may be positioned anywhere on the length of wire inductor 104. In one embodiment, at least one Helmholtz coil spring wire 106 may be positioned in the middle of the length of wire inductor 104. In one embodiment, wire inductor 104 may be made from any material known to one skilled in the art including but not limited to stainless steel, iron, zinc, nickel chromium, high carbon steel, low carbon steel, silver, other alloy material, etc. In one embodiment, wire inductor 104 may have any length known to one skilled in the art. In one embodiment, wire inductor 104 may be cut to any length applicable to one skilled in the art. In one embodiment, wire inductor 104 may be configured to allow trimming allowance for the length required of the finished elongated trombone slide shape and at least one integrated Helmholtz coil loop 106. In one embodiment wire inductor 104 may be trimmed cut to a variable length depending on the length size required to achieve pain relief in the selected appendage area. For example, in one embodiment, when used on a spine lumbar area the length may be about 38 mm and the width may be about 7.0 mm. In one exemplary embodiment, the size for a facial application may be around 20 mm in length and 10 mm in width when using a rectangular electrode coupler. In one exemplary embodiment, when used on a facial area, the size may be an overall 8 mm length and 10 mm width when using a rectangle electrode coupler.

In one embodiment, at least one Helmholtz coil 106 may have at least one turn in the coil. In one embodiment, at least one Helmholtz coil 106 may have a one and one-half turn coil shape. In one embodiment, at least one Helmholtz coil 106 may have any other size known to one skilled in the art. In one embodiment, at least one Helmholtz coil wire inductor loop 106 may be formed using a stationary round steel stem tool sized to the coil circumference size desired. In one embodiment, the coil circumference size may be approximately 3 mm.

In one embodiment, wire inductor 104 with at least one Helmholtz coil 106, when formed, results in two electro-conductive leads positioned at the first end, inserted into the electrode coupler domed slot openings 108. A steel hammer or pressure tool may be struck against the dome slots after the insertion of wire inductor 104 within the slot openings 108, therein completing a closed loop electromagnetic circuit, allowing the attraction of sodium ions into wearable device 100 and dissipating the ions within at least one integrated Helmholtz coil 106. This configuration permits electromagnetic conduction of sodium ions that are attracted to high value Pauling Unit electronegative elements such as gold, silver, stainless steel, iron, nickel chromium, high carbon steel, low carbon steel among other alloys or elements. In one embodiment, electrode coupler base 102 may require slight scuffing of the attachment surface to aid securing wire conductor 104 to the base surface. In another embodiment, wire inductor 104 may be attached to electrode coupler base 102 with any method known to one skilled in the art including but not limited to electroconductive adhesive or solder.

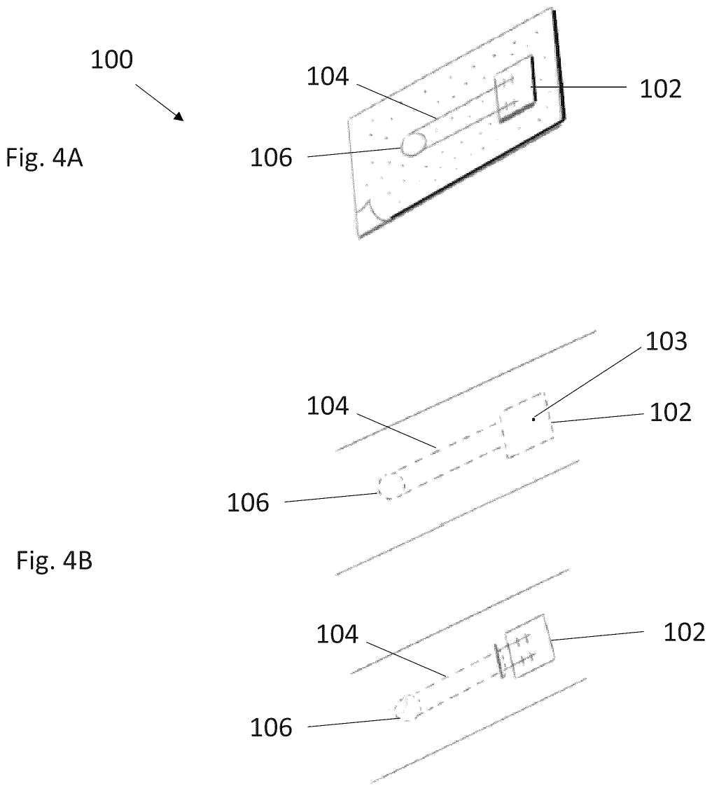

Referring now to FIG. 4A and FIG. 4B, in one embodiment, device 100 may be affixed to the subject by any method known to one skilled in the art including but not limited to adhesive bandage tape, an appendage fabric material, fabric hook and loop belt material, etc. In one embodiment, to affix device 100 to an adhesive bandage tape, the subject may select a length of adhesive bandage tape sufficient to cover the overall length of device 100 and approximately one half inch or more additional allowance on each side to permit securing the tape to the skin (FIG. 4A). In one embodiment, the subject may add an additional allowance of approximately one eighth inch length to provide a small, compressed fold over of the tape to aid in removal of the adhesive tape from the skin. In one embodiment, upon selecting an overall tape length, the subject centers top reverse side 108 of device 100 upon the adhesive side of the tape. Using the lower spine lumber as an example location for placement of device 100, the subject identifies the location of the most painful area and applies the adhesive side of the tape to the external skin. In one embodiment, the subject may apply the device on or near a painful area and then shape it by pressing the sides of the device to the contour of the appendage and if necessary, readjust or reposition as necessary or until the tape loses adhesion.

In one embodiment, device 100 may be affixed to the subject through a use of a fabric material (FIG. 4B). Device 100 may be attached to stretchable fabric material including but not limited to a fabric tube head band by cutting a small horizontal insert slit on one side of the tube fabric and again approximately one half inch longitudinally distal at a location desired. Next, a length of fabric thread is cut and used for securing the device to the tube fabric. The thread is then inserted through the center of at least one Helmholtz coil 106 located inside the fabric material using a sewing needle. Optionally, one or more holes 103 can be drilled into the electrode coupler base 102 to facilitate insertion of the needle. Device 100 is then inserted, through at least one Helmholtz coil 106 end first, into one of the longitudinal slit openings and pushed to the area which will host the securing thread. Obverse side 110 of electrode coupler base 102 of device 100 remains exposed through the first longitudinal slit and contacts the exposed skin when applied by the subject. Alternatively, the subject may expose device 100 externally on the fabric and adhere it without insertion into the fabric tube and then secure the device with fabric thread.

When affixed to external skin or internal organ surfaces causing discomfort, the device of the present invention may relieve pain by attracting sodium ions from nerve cell bodies and prevent pain signal transmission to neuron pathways by attracting sodium ions to the electrode conductor 102 and dissipate sodium ions magnetic fields in the wire inductor 104 Helmholtz coil 106 configuration.

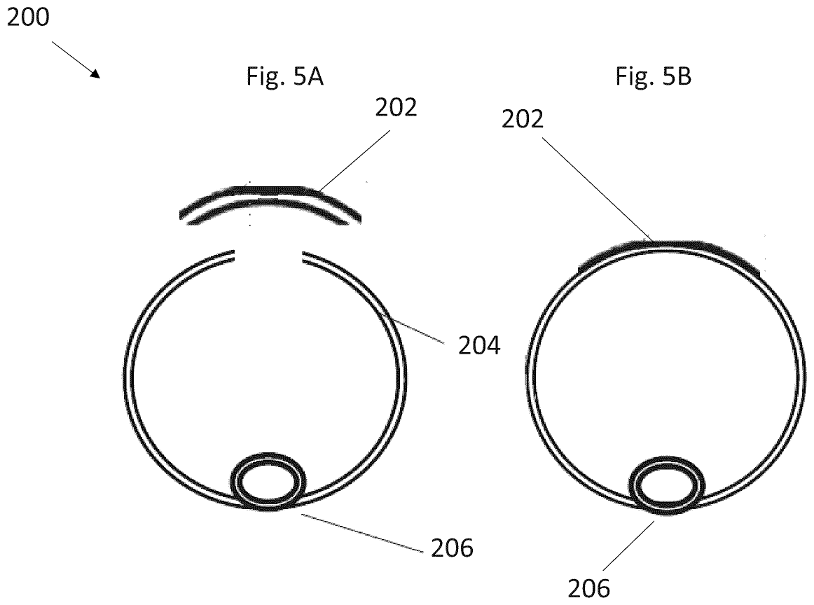

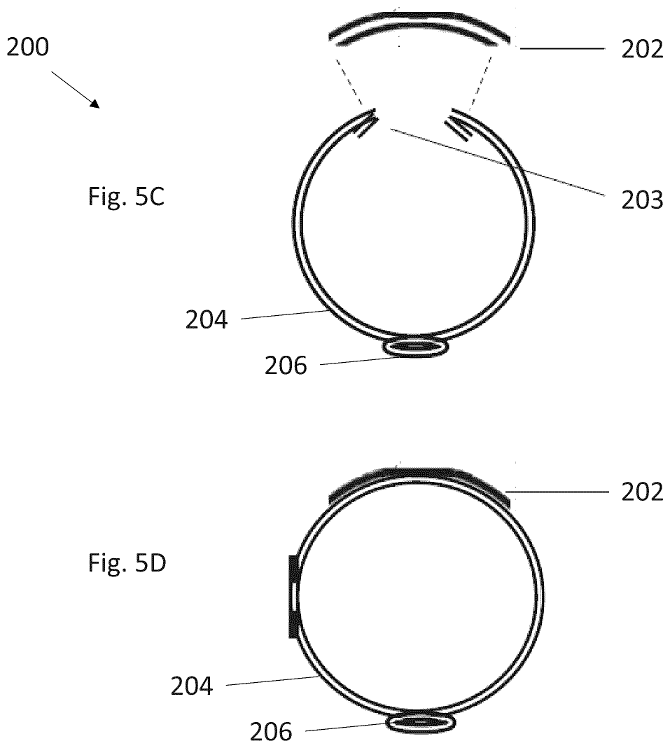

Referring now to FIG. 5A and FIG. 5B, another exemplary pain remediation device 200 of the present invention is shown. Pain remediation device 200 comprises an electrode coupler base 202, a wire inductor 204 and at least one integrated Helmholtz coil 206.

In one embodiment, electrode coupler base 202 may be formed as a hollow curved tube. In one embodiment, electrode coupler base 202 may have two slots at each end. In one embodiment, electrode coupler base 202 may have any other shape known to one skilled in the art. In one embodiment, electrode coupler base 202 may be made from any material known to one skilled in the art including but not limited to alloys of stainless steel, iron, zinc, nickel chromium, high carbon steel, low carbon steel, silver, etc.

In one embodiment, electrode coupler base 202 may have any shape and dimension requiring customization for various pain locations and appendage contours. For example, electrode coupler base 202 may be made from metal to allow customization to provide various bending and stretching based on pain location and appendage contour. In one embodiment, electrode coupler base 202 may be made from light weight metal alloy elements capable of bending and conforming to appendage contours. In one embodiment, electrode coupler base 202 may have compositions with the ability of attracting and conducting electromagnetic fields. In one embodiment, electrode coupler base 202 may be made of materials configured to attract sodium ions. In one embodiment, electrode coupler base 202 may be made of materials that aid in concealability. In one embodiment, electrode coupler base 202 may be made of materials that is comfort for the subject to wear. In one embodiment, electrode coupler base 202 may be obtained and processed by any method known to one skilled in the art including but not limited to laser cutting.

Wire inductor 204 may be a single length round circular shape wire with two electroconductive leads and at least one integrated Helmholtz coil spring wire 206. In one embodiment, at least one Helmholtz coil spring wire 206 may be positioned anywhere on the length of wire inductor 204. In one embodiment, at least one Helmholtz coil spring wire 206 may be positioned in the middle of the length of wire inductor 204. In one embodiment, at least one Helmholtz coil 206 may have at least one turn in the coil. In one embodiment, at least one Helmholtz coil 206 may have any other size known to one skilled in the art.

In one embodiment, wire inductor 204 may be made from any materials known to one skilled in the art including but not limited to stainless steel, iron, zinc, nickel chromium, high carbon steel, low carbon steel, silver, other alloy material, etc. In one embodiment, wire inductor 204 may be made from any material using wire gauges 24 through 18 to allow appendage shape and contour flexibility and provide pain relief while wearing device 200. In one embodiment, wire inductor 204 is light in overall weight providing the subject with enhanced concealability while maintaining utility effectiveness.

In one embodiment, wire inductor 204 may be cut to any length applicable to one skilled in the art. In one embodiment, wire inductor 204 may be formed in a circular ring shape with an integrated Helmholtz circumference of about 3 mm. In one embodiment wire inductor 204 may be trimmed cut to a variable length depending on the length size required to achieve pain relief in the selected appendage area. In one embodiment, wire inductor 204 may be trimmed to an overall size of approximately 23 mm in diameter.

The two leads of wire inductor 204 may be inserted into the two slots, positioned at the two ends of electrode coupler 202. In one embodiment, the two slots may be compressed upon the two leads of wire inductor 204 with compression pressure or hammer tool. In one embodiment, any other method known to one skilled in the art may be used to fix wire inductor 204 and electrode coupler 202 together. In one embodiment, sealant may be added to the two slots of the electrode coupler 202 to prevent ingress of foreign material in the tube.

Referring now to FIG. 5C and FIG. 5D, In one embodiment, wire inductor 204 may further comprise a securing clasp 203 positioned at each end of two electroconductive lead, configured to secure electrode coupler base 202 to wire inductor 204 (FIG. 5C and FIG. 5D). In one embodiment, securing clasp 203 may be 3.2 mm in length. In one embodiment, securing clasp 203 may be shorter than 3.2 mm. In one embodiment, securing clasp 203 may be longer than 3.2 mm. In one embodiment, at least one Helmholtz coil may be horizontally askew.

In one embodiment, device 200 may be suitable to use for finger, toe, or other appendages. In one embodiment, application and shaping of device 200 may be based on the circumference of the finger, toe or other appendage and the breadth of the pain area.

Device 200 is configured to permit the attraction of sodium ions into the device, and dissipates ions therein, when affixes to the skin or positioned near a painful area.

Device 200 may be positioned in place in a similar manner described above for device 100.

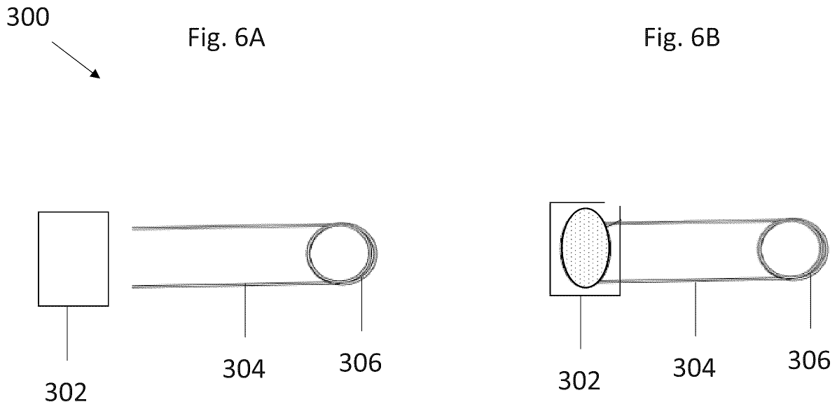

Referring now to FIG. 6A and FIG. 6B, another exemplary pain remediation device 300 of the present invention is shown. Pain remediation device 300 comprises an electrode coupler base 302, a two lead wire inductor 304 and at least one integrated Helmholtz coil 306.

In one embodiment, electrode coupler base 302 may be made from any material known to one skilled in the art including but not limited to alloys of stainless steel, iron, zinc, nickel chromium, high carbon steel, low carbon steel, silver sheets, etc. In one embodiment, electrode coupler base 302 may have a gauge size ranging between 30 through 18. In one embodiment, electrode coupler base 302 may have a thickness ranging between 0.318-1.219 mm. In one embodiment, electrode coupler base 302 may have an overall dimension ranging between 9.525-12.7 mm square shape. In one embodiment,

electrode coupler base 302 may have an overall dimension of 12 mm in length and 9 mm in width. In one embodiment, the outer corners of electrode coupler base 302 may be beveled or rounded to avoid sharp edges against the subject's skin. In one embodiment, final shaping of the device may be achieved by flattening electrode coupler base 102 and reverting flat any unwanted curvatures using pressure blows by a steel hammer or pressure devices and alternatively using a sanding grinder or file as necessary to remove sharp edges. In one embodiment, electrode coupler base 302 may be subjected to base size cutting, edge smoothing and rounding.

In one embodiment, electrode coupler base 302 may have any shape known to one skilled in the art including but not limited to arched, round, rectangle, square, keystone, flat, curve, hollow curve tube, or straight tube shapes. In one embodiment, electrode coupler base 302 may have any shape and dimension requiring customization for various pain locations and appendage contours. For example, electrode coupler base 302 may be made from metal to allow customization to provide various bending and stretching based on pain location and appendage contour. In one embodiment, electrode coupler base 302 may be made from light weight metal alloy elements capable of bending and conforming to appendage contours. In one embodiment, electrode coupler base 302 may have compositions with the ability of attracting and conducting electromagnetic fields. In one embodiment, electrode coupler base 302 may be made of materials configured to attract sodium ions. In one embodiment, electrode coupler base 302 may be made of materials that aid in concealability. In one embodiment, electrode coupler base 302 may be made of materials that is comfort for the subject to wear. In one embodiment, electrode coupler base may be any dimension sizes to accommodate customization when required for specialized use such as in facial applications to aid in concealability.

In one embodiment, electrode coupler base 302 may be obtained and processed by any method known to one skilled in the art including but not limited to laser cutting.

Wire inductor 304 is a single length wire inductor with two electroconductive leads at a first end and at least one integrated Helmholtz coil spring wire 306 at a second end. In one embodiment, at least one Helmholtz coil spring wire 306 may be positioned anywhere on the length of wire inductor 304. In one embodiment, at least one Helmholtz coil spring wire 306 may be positioned in the middle of the length of wire inductor 304. In one embodiment, wire inductor 304 may be made from any material known to one skilled in the art including but not limited to stainless steel, iron, zinc, nickel chromium, high carbon steel, low carbon steel, silver, other alloy material, etc. In one embodiment, wire inductor 304 may have any length known to one skilled in the art. In one embodiment, wire inductor 304 may be cut to any length applicable to one skilled in the art. In one embodiment, wire inductor 304 may be configured to allow trimming allowance for the length required of the finished elongated trombone slide shape and at least one integrated Helmholtz coil loop 306. In one embodiment wire inductor 304 may be trimmed cut to a variable length depending on the length size required to achieve pain relief in the selected appendage area. In one exemplary embodiment, when used on a facial area, the size may be an overall 8 cm length and 1.0 mm width when using a rectangle electrode coupler 302.

In one embodiment, at least one Helmholtz coil 306 may have at least one turn in the coil. In one embodiment, at least one Helmholtz coil 306 may have a one and one-half turn coil shape. In one embodiment, at least one Helmholtz coil 306 may have any other size known to one skilled in the art. In one embodiment, at least one Helmholtz coil wire inductor loop 306 may be formed using a stationary round steel stem tool sized to the coil circumference size desired. In one embodiment, the coil circumference size may be approximately 3 mm. In one embodiment, the coil circumference size may be more than 3 mm. In one embodiment, the coil circumference size may be less than 3 mm.

In one embodiment, wire inductor 304 may be attached to electrode coupler base 302 with any method known to one skilled in the art including but not limited to electroconductive adhesive or solder. In one embodiment, electrode coupler base 302 may require slight scuffing of the attachment surface to aid securing wire conductor 304 to the base surface. In one embodiment, at least one integrated Helmholtz coil 306, when formed may result in two conductive leads that are adhesively applied to the scuffed surface of the electrode coupler base 302 using a electro-conductive high strength epoxy adhesive, metal alloy solder, or Bondic [TM] Ultraviolet activated glue to complete the electromagnetic conductive closed loop circuit assembly. In one embodiment, device 300 may be affixed to skin on or near a painful area and is configured to attract sodium ions in the device assembly and dissipate the ions in at least one Helmholtz coil 306.

Device 300 may be positioned in place in a similar manner described above for device 100. In one embodiment, device 300 may be used on facial areas to provide dental pain relief. In one embodiment, device 300 may be used on any other body locations. In one embodiment, the subject identifies the location of the most painful area and applies the obverse side of the device and adhesive tape to the skin above or near a painful area. In one embodiment, the subject may repeat the process to add a second device above or below the first application if necessary. In one embodiment, the subject may easily remove the device attached to the adhesive bandage tape and readjust the location as necessary until the tape loses adhesion.

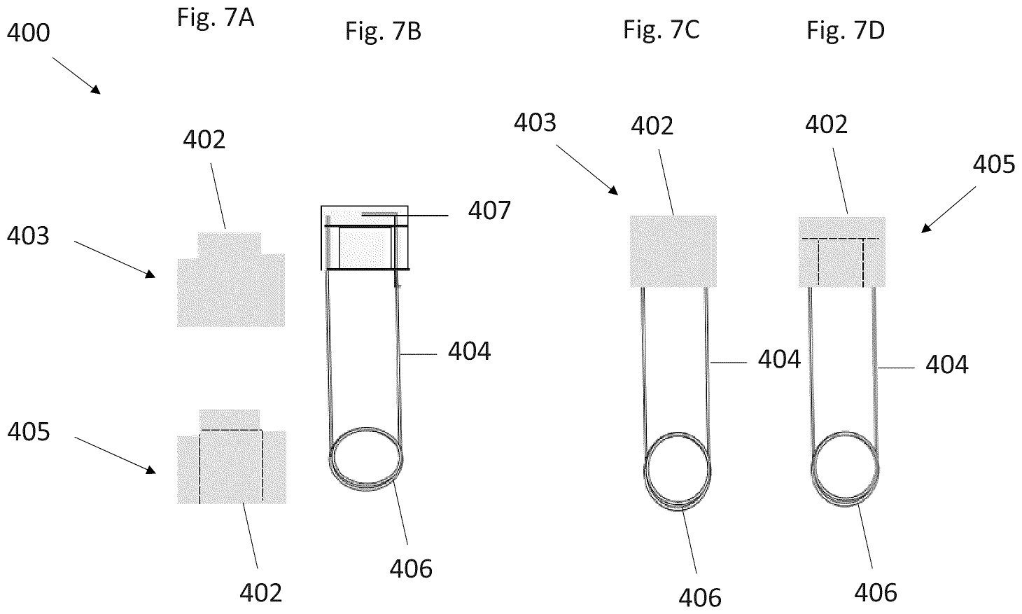

Referring now to FIG. 7A through FIG. 7D, another exemplary pain remediation device 400 of the present invention is shown. Pain remediation device 400 comprises an electrode coupler base 402, a two lead wire inductor 404 and at least one integrated Helmholtz coil 406.

Electrode coupler base 402 comprises a obverse side 403 and an reverse side 405. In one embodiment, electrode coupler base 402 may be made with electromagnetic conductive alloy materials including but not limited to stainless steel, iron, zinc, nickel chromium, high carbon steel, low carbon steel, or silver alloy material. Electrode coupler base 402 may have a keystone shape, with three column shape with two columns having 90 degree square cutouts that form three folding hem sides, configured to permit insertion and compression of wire inductor 404.

In one embodiment, electrode coupler base 402 may have a gauge size ranging between 30 through 24. In one embodiment, electrode coupler base 402 may be made of stainless steel, iron, zinc, nickel chromium, high carbon steel, low carbon steel, or silver sheet material. In one embodiment, electrode coupler base 402 may be 19 mm square before two, 4 mm ninety degree square cuts are made to the left and right side of the initial square metal sheet coupler.

Wire inductor 404 is a single length wire inductor with two electroconductive leads at a first end and at least one integrated Helmholtz coil spring wire 406 at a second end. In one embodiment, at least one Helmholtz coil spring wire 406 may be positioned anywhere on the length of wire inductor 404. In one embodiment, at least one Helmholtz coil spring wire 406 may be positioned in the middle of the length of wire inductor 404.

In one embodiment, at least one Helmholtz coil 406 may have at least one turn in the coil. In one embodiment, at least one Helmholtz coil 406 may have a one and one-half turn coil shape. In one embodiment, at least one Helmholtz coil 406 may have any other size known to one skilled in the art. In one embodiment, at least one Helmholtz coil wire inductor loop 406 may be formed using a stationary round steel stem tool sized to the coil circumference size desired. In one embodiment, the coil circumference size may be approximately 3 mm. In one embodiment, the coil circumference size may be more than 3 mm. In one embodiment, the coil circumference size may be less than 3 mm.

In one embodiment, wire inductor 404 may be made from any material known to one skilled in the art including but not limited to stainless steel, iron, zinc, nickel chromium, high carbon steel, low carbon steel, silver, other alloy material, etc. to maximize the conductivity of wire inductor 404. In one embodiment, wire inductor 404 may have any length known to one skilled in the art. In one embodiment, wire inductor 404 may be cut to any length applicable to one skilled in the art. In one embodiment, wire inductor 404 may comprise a sharp ninety degree bend 407 on one electroconductive lead configured for securing wire inductor 404 to electrode coupler base 402. In one embodiment, wire inductor 404 may be configured to allow trimming for the length of the finished device including at least one Helmholtz coil loop 406 and a sharp ninety degree left return bend 407 that is used for securing the said wire inductor to the said electrode coupler base. In one embodiment wire inductor 404 may be trimmed cut to a variable length depending on the length size required to achieve pain relief in the selected appendage area.

In one embodiment wire inductor 404 may be trim cut to a variable length plus also providing an extra allowance for the sharp ninety degree left bend 407. In one embodiment, wire inductor leads 404 may be inserted into the reverse side 405 of electrode coupler hemmed flaps and compressed to secure electrode coupler 402 to wire inductor 404 to complete a closed loop electromagnetic circuit when applied to skin. In one embodiment, a steel hammer or pressure tool is used to compress the inserted wire inductor 404 under the hemmed flaps of the electrode coupler base 402. Device 400, when affixed to skin creates a closed loop electromagnetic circuit to attract sodium ions into the therapeutic device assembly and dissipate the ions in at least one Helmholtz coil 406.

Device 400 may be positioned in place in a similar manner described above for device 100.

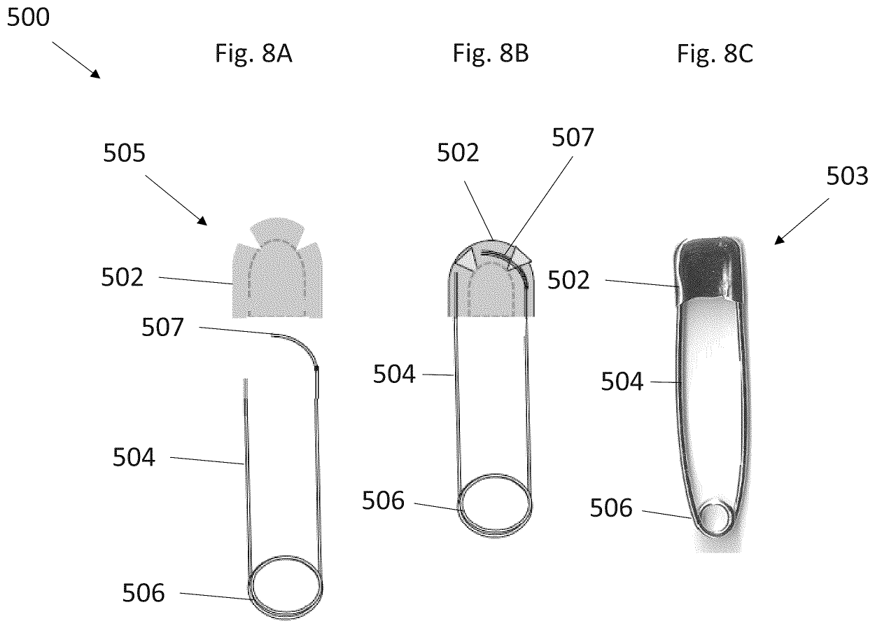

Referring now to FIG. 8A through FIG. 8C, another exemplary pain remediation device 500 of the present invention is shown. Pain remediation device 500 comprises an electrode coupler base 502, a two lead wire inductor 504 and at least one integrated Helmholtz coil 506.

Electrode coupler base 502 comprises a obverse side 503 and an reverse side 505. In one embodiment, electrode coupler base 502 may be made from any material known to one skilled in the art including but not limited to alloys of stainless steel, iron, zinc, nickel chromium, high carbon steel, low carbon steel, silver sheets or wire, etc. In one embodiment, electrode coupler base 502 may be fabricated with any method known to one skilled in the art including but not limited to a laser cutter. In one embodiment, electrode coupler base 502 may be shaped as an arched half round and half rectilinear with two triangular shape cutouts that are used as compression hems. In one embodiment, electrode coupler base 502 may have a metal gauge size ranging between 30 through 24. In one embodiment, electrode coupler base 502 may have an overall dimension of 16 mm length by 12 mm width. In one embodiment, electrode coupler base 502 may have an arched top shape and two triangular compression fold configured to secure wire inductor 504.

Wire inductor 504 is a single length wire inductor with two electroconductive leads at a first end and at least one integrated Helmholtz coil spring wire 506 at a second end. In one embodiment, at least one Helmholtz coil spring wire 506 may be positioned anywhere on the length of wire inductor 504. In one embodiment, at least one Helmholtz coil spring wire 506 may be positioned in the middle of the length of wire inductor 504. In one embodiment, at least one Helmholtz coil 506 may have at least one turn in the coil. In one embodiment, at least one Helmholtz coil 506 may have a one and one-half turn coil shape. In one embodiment, at least one Helmholtz coil 506 may have any other size known to one skilled in the art. In one embodiment, at least one Helmholtz coil wire inductor loop 506 may be formed using a stationary round steel stem tool sized to the coil circumference size desired. In one embodiment, the coil circumference size may be approximately 3 mm. In one embodiment, the coil circumference size may be more than 3 mm. In one embodiment, the coil circumference size may be less than 3 mm.

In one embodiment, wire inductor 504 may be made from any material known to one skilled in the art including but not limited to stainless steel, iron, zinc, nickel chromium, high carbon steel, low carbon steel, silver, other alloy material, etc. In one embodiment, wire inductor 504 may be cut to any length applicable to one skilled in the art. In one embodiment, wire inductor 504 may comprise a left facing arch radius return bend 507, creating two lead wires which are secured to electrode coupler base 502. In one embodiment, wire inductor 504 may run descending vertically 30 mm with a formed 3 mm circumference one and one half turn Helmholtz coil and a parallel return run of spring wire 35 mm encompassing a arching left radius bend 507 as a return run towards the first vertical descending spring wire which will then be inserted and secured under reverse side 505 of the electrode coupler hem flaps and compressed.

In one embodiment, the final assembly of device 500 when affixed to skin creates a closed loop electromagnetic circuit to attract sodium ions into the assembled device and dissipates the ions in at least one Helmholtz coil 506.

Device 500 may be positioned in place in a similar manner described above for device 100.

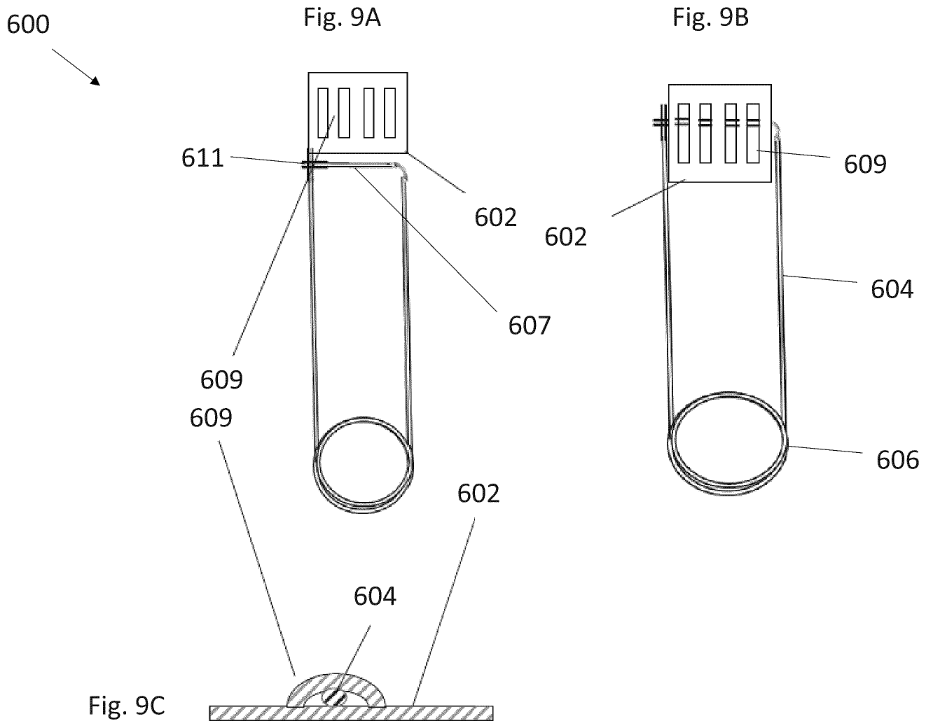

Referring now to FIG. 9A through FIG. 9C, another exemplary pain remediation device 600 of the present invention is shown. Pain remediation device 600 comprises an electrode coupler base 602, a two lead wire inductor 604 and at least one integrated Helmholtz coil 606.

Electrode coupler base 602 may have any shape known to one skilled in the art including but not limited to a square. In one embodiment, electrode coupler base 602 may be made from any material known to one skilled in the art including but not limited to alloys of stainless steel, iron, zinc, nickel chromium, high carbon steel, low carbon steel, silver sheets or wire, etc. In one embodiment, electrode coupler base 602 may be fabricated with any method known to one skilled in the art including but not limited to a laser cutter. In one embodiment, electrode coupler base 602 may have a gauge size ranging between 30 through 22. In one embodiment, electrode coupler base 602 may have an overall dimension of 9.525 mm by 9.525 mm square shape.

In one embodiment, electrode coupler base 602 comprises four slot openings 609. In one embodiment, four slot openings 609 may have any known shapes to one skilled in the art including but not limited to a square, oval, rectangular, etc. In one embodiment, four slot openings 609 may have a vertically oriented rectangular shape. In one embodiment, two slot openings 609 may be dome shaped. In one embodiment, dome shape openings may be made with any method known to one skilled in the art including but not limited to a dapple doming tool. In one embodiment, dapple doming tool is applied upon the center solid metal column of the left side two slot openings 609 and a second dome bridge is formed between the right side two slot openings 609.

In one embodiment, the dome bridge slot openings 609 may be formed by centering one of the electrode coupler base bridge columns over a 9.1 mm hole drilled into a hardened metal sheet of sufficient thickness, typically 3.125 mm to allow pressure bending of the dome bridge insertion slot openings 609 with the dapple doming tool without tearing or shearing electrode coupler base 602 and using repeated strikes by a steel hammer or other pressure bending tool or machine.

Wire inductor 604 is a single length wire inductor with two electroconductive leads at a first end and at least one integrated Helmholtz coil spring wire 606 at a second end. In one embodiment, at least one Helmholtz coil spring wire 606 may be positioned anywhere on the length of wire inductor 604. In one embodiment, at least one Helmholtz coil spring wire 606 may be positioned in the middle of the length of wire inductor 604. In one embodiment, at least one Helmholtz coil 606 may have at least one turn in the coil. In one embodiment, at least one Helmholtz coil 606 may have a one and one-half turn coil shape. In one embodiment, at least one Helmholtz coil 606 may have any other size known to one skilled in the art. In one embodiment, at least one Helmholtz coil wire inductor loop 606 may be formed using a stationary round steel stem tool sized to the coil circumference size desired. In one embodiment, the coil circumference size may be approximately 5 mm. In one embodiment, the coil circumference size may be less than 5 mm. In one embodiment, the coil circumference size may be more than 5 mm.

In one embodiment, wire inductor 604 may be made from any material known to one skilled in the art including but not limited to stainless steel, iron, zinc, nickel chromium, high carbon steel, low carbon steel, silver, other alloy material, etc. In one embodiment, wire inductor 604 may have any length known to one skilled in the art. In one embodiment, wire inductor 604 may be cut to any length applicable to one skilled in the art.

Wire inductor 604 comprises a securing clasp having a sharp left 90 degree return bend 607 and a one 180 degree wrap around clasp 611 for securing electrode base coupler 602 to wire inductor 604. In one embodiment, one electroconductive lead of spring wire material is first run descending vertically about 38 mm, then forming Helmholtz coil 606 and a parallel return run of spring wire of 48 mm, bending sharply 90 degrees left as a return run towards the first electroconductive lead. Wire inductor 604 is then inserted through the two electrode coupler bridge dome slot openings 609 and bent 180 degrees around the outside of the first descending vertical spring wire which is used as a stabilizing clasp to secure electrode coupler 602 to wire inductor 604.

Upon insertion, electrode coupler bridge dome shaped slot openings 609 may be compressed by a pressure device such as a steel hammer to further secure wire inductor 604 to electrode coupler 602. In one embodiment, electrode coupler base 602 can be additionally secured with waterproof adhesive glue to prevent the ingress of foreign material into slot openings 609. In one embodiment, final shaping of the therapeutic device can be achieved by flattening and smoothing sharp edges. In one embodiment, the final assembly of device 600, when affixed to skin creates a closed loop electromagnetic circuit to attract sodium ions into device 600 and dissipate the ions in at least one Helmholtz coil 606.

Device 600 may be positioned in place in a similar manner described above for device 100.

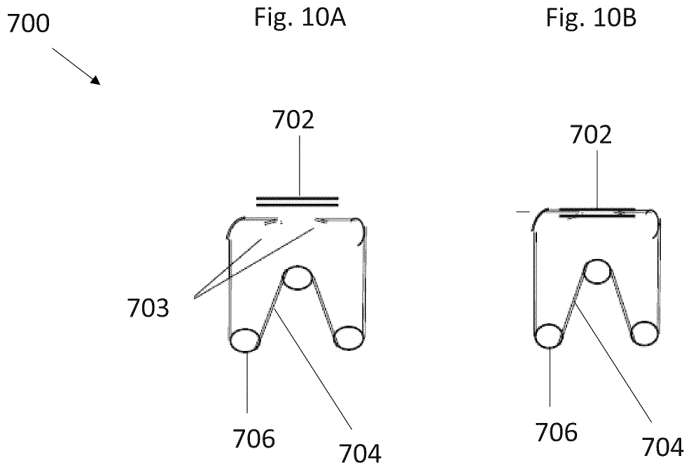

Referring now to FIG. 10A and FIG. 10B, another exemplary pain remediation device 700 of the present invention is shown. Pain remediation device 700 comprises an electrode coupler base 702, a two lead wire inductor 704 and three integrated Helmholtz coil 706.

In one embodiment, electrode coupler base 702 may be formed as a hollow straight tube. In one embodiment, electrode coupler base 702 may have two slots at each end. In one embodiment, electrode coupler base 702 may have any other shape known to one skilled in the art. In one embodiment, electrode coupler base 702 may be made from any material known to one skilled in the art including but not limited to alloys of stainless steel, iron, zinc, nickel chromium, high carbon steel, low carbon steel, silver, etc.

Wire inductor 704 may be a single length wire with having multiple horizontal and vertical angles with two electroconductive leads at the ends and three integrated Helmholtz coil spring wire 706. In one embodiment, wire inductor 704 may be made from any material known to one skilled in the art including but not limited to alloys of stainless steel, iron, zinc, nickel chromium, high carbon steel, low carbon steel, silver, etc.

In one embodiment, the material for wire inductor 704 can be hard or semi soft spring wire. In one embodiment, wire inductor 704 may be formed on the left side with a bent 180 degrees securing clasp of approximately 3.2 mm for insertion into the ends of electrode coupler base straight tube 702 and extended horizontally 9.5 mm length, then bent to a ninety degree radius and extended vertically downward typically 20 mm encompassing a counterclockwise one turn Helmholtz coil then angled vertically upward 45 degrees and extended vertically 13 mm encompassing a clockwise one turn Helmholtz coil and then angled vertically downward 45 degrees and extended 13 mm encompassing a counterclockwise one turn Helmholtz coil and then angled upward 90 degrees and extended vertically 20 mm length encompassing a ninety degree radius and extended horizontally 9.5 mm with a 3.2 mm clasp end bent 180 degrees to form a second securing clasp inserted on the right side of the electrode base coupler 702. In one embodiment, the lengths outlined above may be longer or shorter based on the application. In one embodiment, at least three Helmholtz coils 706 may be positioned anywhere along the length of wire inductor 704.

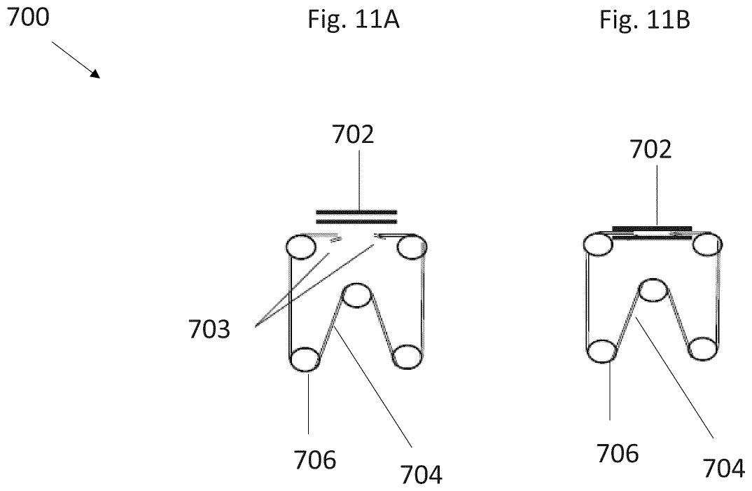

Referring now to FIG. 11A and FIG. 11B, device 700 may have at least five integrated Helmholtz coil 706. In one exemplary embodiment, wire inductor 704 formed on the left side with a bent 180 degrees securing clasp 703 of approximately 3.2 mm for insertion into the straight tube electrode coupler base 702 and extended horizontally 9.5 mm length, encompassing a counterclockwise one turn Helmholtz coil loop then extended downward vertically typically 20 mm encompassing a counterclockwise one turn Helmholtz coil then angled upward 45 degrees and extended vertically 13 mm encompassing a clockwise one turn Helmholtz coil and exiting then angled downward 45 degrees and extended vertically 13 mm encompassing a counterclockwise one turn Helmholtz coil and exiting then angled upward and extended vertically 20 mm length encompassing a counterclockwise one turn Helmholtz coil loop exiting and extended horizontally 9.5 mm with a 3.2 mm end bent 180 degrees to form a second securing clasp 703 within the electrode coupler right side.

In one embodiment, the left and right horizontal return wire inductors form an anchoring clasp 703, attached to each end of electrode coupler base 702. Electrode coupler base 702 is then compressed with a hammer or pressure tool to secure the coupler to wire inductor 704. In one embodiment, the final assembly of device 700 when affixed to skin creates a closed loop electromagnetic circuit to attract sodium ions into the device and dissipate the ions in Helmholtz coils 706.

Device 700 may be positioned in place in a similar manner described above for device 100.

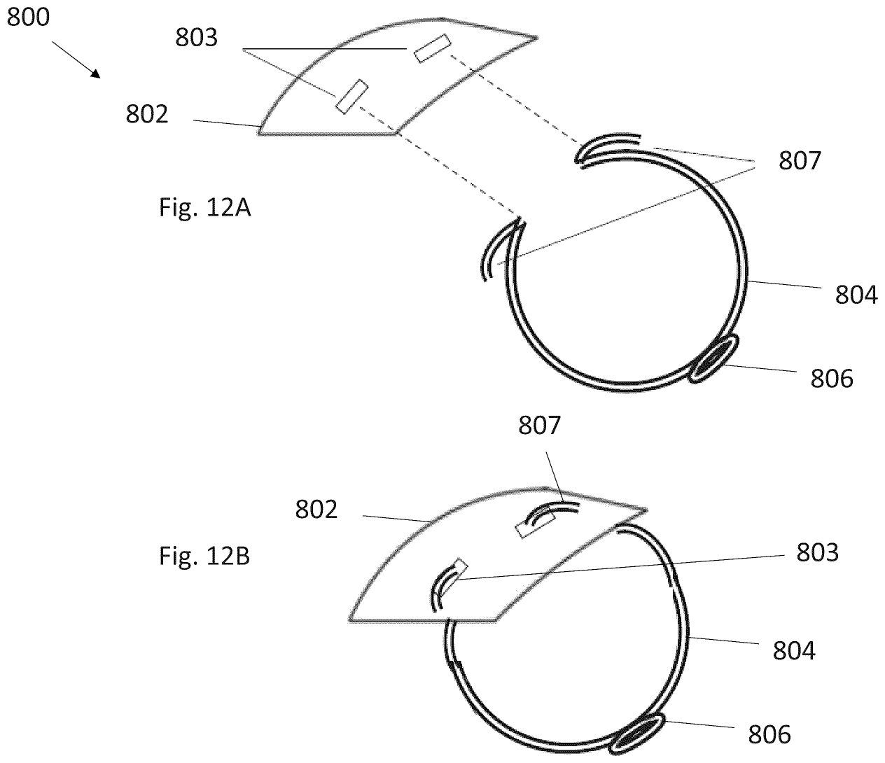

Referring now to FIG. 12A through FIG. 12B, another exemplary pain remediation device 800 of the present invention is shown. Pain remediation device 800 comprises an electrode coupler base 802, a two lead wire inductor 804 and at least one integrated Helmholtz coil 806.

In one embodiment, electrode coupler base 802 may be made from any material known to one skilled in the art including but not limited to alloys of stainless steel, iron, zinc, nickel chromium, high carbon steel, low carbon steel, silver, etc. In one embodiment, electrode coupler base 802 may have any shape known to one skilled in the art including but not limited to arched, round, rectangle, square, keystone, flat, curve, hollow curve tube, or straight tube shapes. In one embodiment, electrode coupler base 802 may be rectangular or square in shape. Electrode coupler base 802 may have two slot openings 803 configured to be used for inserting the leads of wire inductor 804. The two leads of wire inductor 804 with at least one integrated Helmholtz coil 806 may be inserted each into either side of slot openings 803 and folded outward on the reverse side of electrode coupler 802 and compressed to secure wire inductor 804 to electrode coupler base 802. In one embodiment, assembled device 800 may be used as ring design to fit a finger, toe or other appendage. In one embodiment, this design permits flexibility to allow the fold over securing the two leads of wire inductor 804 to slide along the reverse side slots 803 of electrode coupler surface 802, allowing the ring to also slide over swollen joints then permitting skin contact of the appendage skin.

In one embodiment, electrode coupler base 802 may be made from metal sheets having a gauge size ranging between 30 through 24. In one embodiment, electrode coupler base 802 may have an overall dimension of 9.525 mm by 9.525 mm. In one embodiment, electrode coupler base 802 may have an overall dimension of less than 9.525 mm by 9.525 mm. In one embodiment, electrode coupler base 802 may have an overall dimension of 9.525 mm by 11 mm for rectangular shapes. In one embodiment, electrode coupler base 802 may have an overall dimension of less than 9.525 mm by 11 mm for rectangular shapes.

In one embodiment, at least one Helmholtz coil 806 may be positioned anywhere on the length of wire inductor 804. In one embodiment, at least one Helmholtz coil 806 may be positioned in the middle of the length of wire inductor 804. In one embodiment, wire inductor 804 may have any length known to one skilled in the art. In one embodiment, wire inductor 804 may be cut to any length applicable to one skilled in the art. In one embodiment, wire inductor 804 may be configured to allow trimming allowance for the length required of the finished ring shape and at least one integrated Helmholtz coil loop 806. In one embodiment wire inductor 804 may be trimmed cut to a variable length depending on the length size required to achieve pain relief in the selected appendage area.

In one embodiment, at least one Helmholtz coil 806 may have at least one turn in the coil. In one embodiment, at least one Helmholtz coil 806 may have a one and one-half turn coil shape. In one embodiment, at least one Helmholtz coil 806 may have any other size known to one skilled in the art. In one embodiment, at least one Helmholtz coil wire inductor loop 806 may be formed using a stationary round steel stem tool sized to the coil circumference size desired. In one embodiment, the coil circumference size may be approximately 4 mm. In one embodiment, the coil circumference size may be smaller than 4 mm. In one embodiment, at least one Helmholtz coil 806 may be bent askew horizontally ninety degrees relative to skin surface. In one embodiment, at least one Helmholtz coil 806 may be bent askew to any other degrees less than 90 degree relative to skin surface.

In one embodiment, wire inductor 804 may further comprise a securing clasp 807 positioned at each end of two electroconductive lead configured to secure electrode coupler base 802 to wire inductor 804. In one embodiment, securing clasp 807 may be approximately 4 mm in length. In one embodiment, securing clasp 807 may be shorter than 4 mm. In one embodiment, securing clasp 807 may be longer than 4 mm. In one embodiment, two electroconductive lead tips are inserted in one each of the two rectangular slot openings 803 of electrode coupler 802 and the lead tips are folded back against the electrode coupler and compressed with a compression pressure or steel hammer tool to secure device 800 assembly and to shape to the appendage contour. In one embodiment, assembled device 800, when affixed to the skin on or near a painful area, permits the attraction of sodium ions into the therapeutic device, and dissipates the ions in at least one Helmholtz coil 806.

Device 800 may be positioned in place in a similar manner described above for device 100.

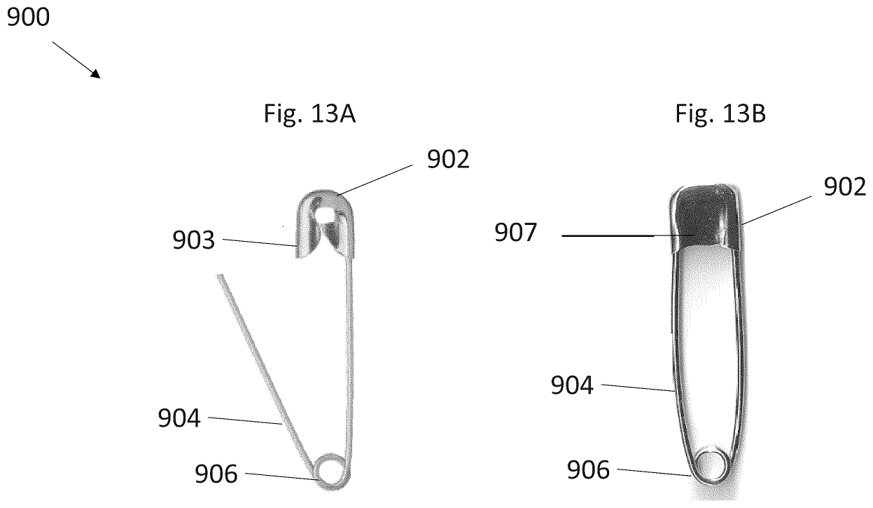

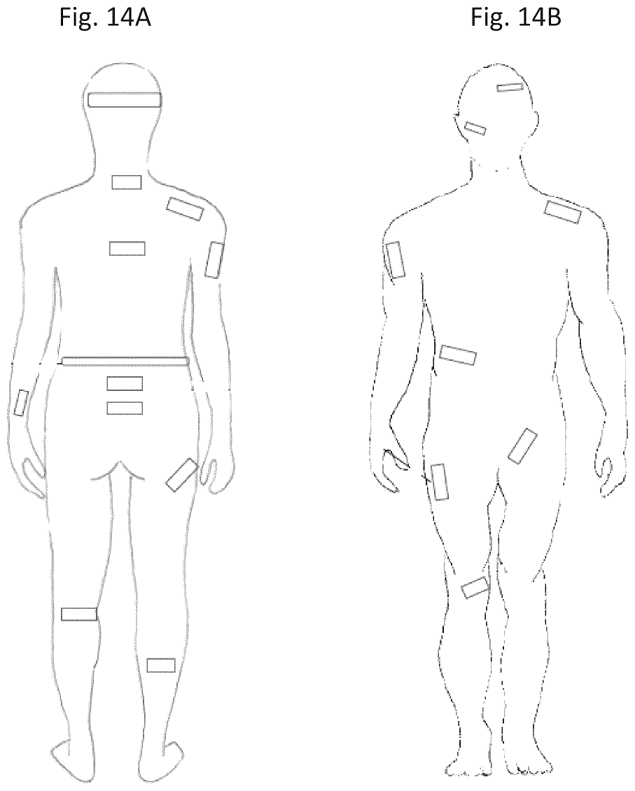

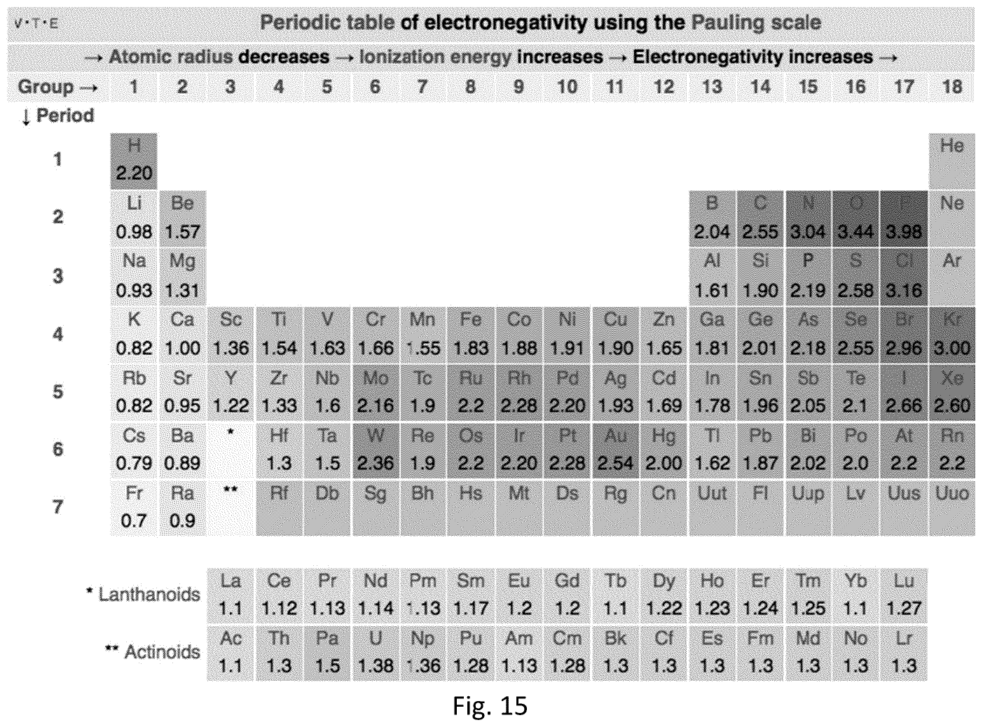

Referring now to FIG. 13A and FIG. 13B, another exemplary pain remediation device 900 of the present invention is shown. Pain remediation device 900 comprises an electrode coupler base 902, a two lead wire inductor 904 and at least one integrated Helmholtz coil 906.

In one embodiment, electrode coupler 902 may have a slotted arched half round shape and a half rectilinear shape closure electrode coupler configured to allow locking of wire inductors and closing the circuit. In one embodiment, electrode coupler 902 comprises a clasp design 903 in a shape of a safety pin. In one embodiment, device 900 includes improvement designs for safety to prevent accidental wire detachment stabbing and skin abrasion when using the device for purposes of pain relief.

The two leads of wire inductor 904 with at least one integrated Helmholtz coil 906 may be made from semi hard or hard spring wire having an elongated trombone slide shape with two parallel attachment leads with at least one integrated Helmholtz coil design on a common axis. In one embodiment, device 900 is assembled to a slotted half rounded and half rectilinear folded sheet metal electrode coupler clasp 903 having an arching slotted radius clasp to secure the two lead wire inductor 904 and together now forming a passive electromagnetic closed loop wearable therapeutic pain remediation device to attract sodium ions and dissipate said ions in at least one Helmholtz coil 906. In one embodiment, to enhance safety for affixing to human skin, an electromagnetic adhesive 907 may be added within the slotted area of the electrode coupler security clasp 903 to prevent accidental wire detachment stabbing and abrasion of skin when using device 900 on human skin to provide pain relief.

In one embodiment, electrode coupler base 902 may be made from any material known to one skilled in the art including but not limited to alloys of stainless steel, iron, zinc, nickel chromium, high carbon steel, low carbon steel, silver, etc. In one embodiment, electrode coupler base 902 may be made with metal gauge sizes ranging between 20 through 38 gauge. In one embodiment, the use of a spring wire inductor 904 and security clasp 903 known generically as a safety pin may function as a safer, low cost wearable therapeutic pain remediation device 900 when adhered to skin. In one embodiment, device 900 may permit the attraction of sodium ions into the therapeutic device and dissipate ions in at least one Helmholtz coil 906.

In one embodiment, at least one Helmholtz coil 906 may have at least one turn in the coil. In one embodiment, at least one Helmholtz coil 906 may have a one and one-half turn coil shape. In one embodiment, at least one Helmholtz coil 906 may have any other size known to one skilled in the art. In one embodiment, at least one Helmholtz coil wire inductor loop 906 may be formed using a stationary round steel stem tool sized to the coil circumference size desired. In one embodiment, the coil circumference size may be approximately 3 mm. In one embodiment, the coil circumference size may be smaller than 3 mm.

Device 900 may be positioned in place in a similar manner described above for device 100.

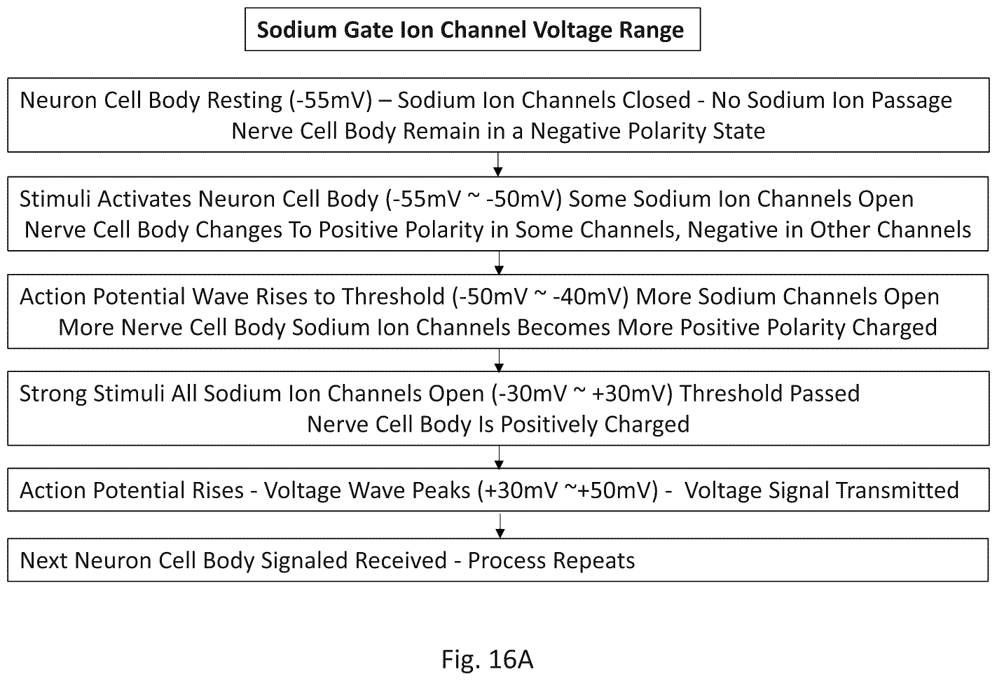

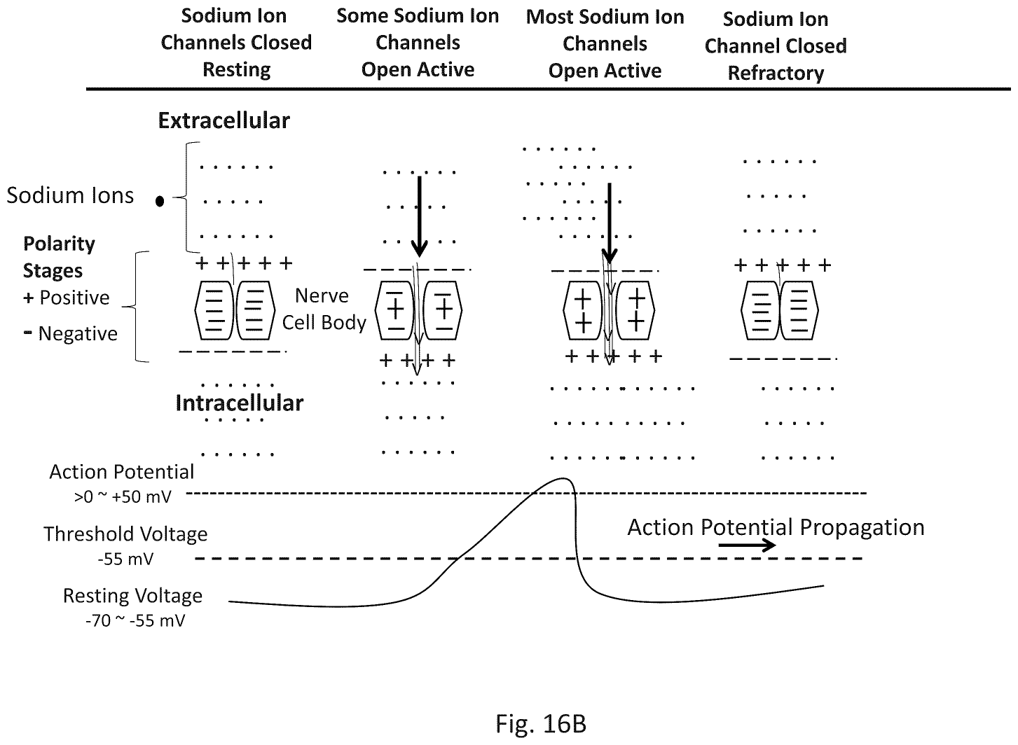

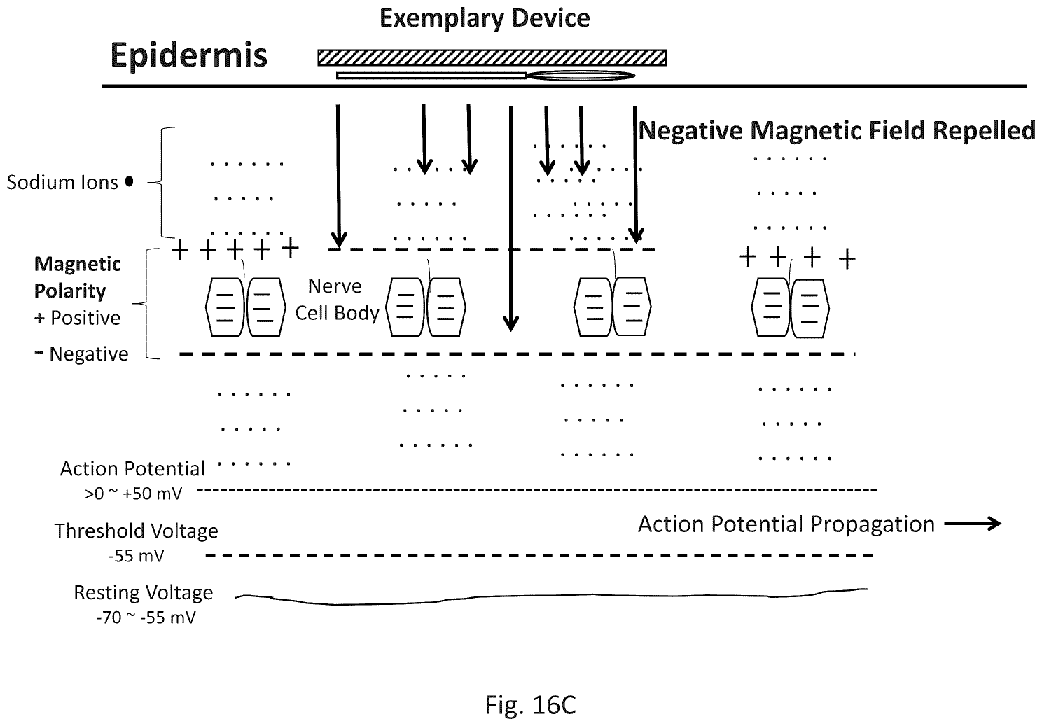

Referring now to FIG. 16A, FIG. 16B and FIG. 16C, sodium gate ion channel voltage range and function is shown. The neuron cell body is an excitable cell that forms an electromagnetic voltage wave created within the nerve cell body by the passage of positive charged sodium and potassium ions forming an action potential wave form that transmits stimuli information from one cell body to another along a nerve pathway. The extracellular space located above the nerve cell body has positively charge sodium ions, and the intercellular space below the nerve cell body has electronegative and electropositive sodium ion charges. Three types of ions, calcium, potassium, and sodium, have different specific atomic size ion channels within the nerve cell bodies that open when conditions have been met to allow only specific atomic size ions to pass through generating electrochemical voltage action potentials in the nerve cell body. The electropositive sodium ions atomic size is smaller than electronegative sodium ions because electropositive sodium ions donate electrons to electronegative sodium ions increasing its size. The smaller electropositive sodium ions passing through gated tubes in the cell body create a voltage charge and change the intercellular space positive creating a magnetic and electric field charge permitting the creation of a voltage action potential.

The nerve cell body has resting, active, and inactive states (FIG. 16B). During each phase the channel tube is blocked, passable (some channels open) or more passible (most channels open) for the electropositive sodium ion and the number of open channels are dependent on the stimulus intensity. Sodium ion channels in the nerve cell body are responsible for the rising phase of a voltage action potential to transmit signals.

In a resting state, a nerve cell body has closed sodium ion channels that are blocked, and no electropositive sodium ions pass through keeping the nerve cell body and the intercellular space in a negative voltage state charge (−55 mV). In a stimuli active state, the nerve cell body becomes excitable and some electropositive sodium ion channels begin unblocking allowing some electropositive sodium ions to pass changing the magnetic polarity of the nerve cell body and the intercellular space more positively charged allowing a voltage waveform to begin rising to a set threshold voltage charge (>−55 mV), permitting an action potential waveform to continue rising.

With stronger stimuli, most electropositive sodium ion channels open turning the nerve cell body and the intercellular space magnetic polarity positive and raising the voltage above the threshold (>−55 mV) in the nerve cell body to a peak voltage typically greater than o volts to a peak (+50 mV). At this voltage an electrical action potential is created to transmit the waveform to the next nerve cell body along the pathway. When a number of nerve cell bodies activate, a voltage waveform propagates the neuronal pathway transmitting the signal to the central nervous system. After transmitting the voltage action potential, the nerve cell body close the sodium ion channels and the potassium ion channels open to return the nerve cell body back to a negative polarity resting state (−77 mV) and the intercellular space also return to a negative polarity space. The outer cellular space returns to a positive polarity state.

In one embodiment, the purpose of the exemplary devices described above, located on the skin is to attract donating electropositive sodium ions to attractive metals in the device and keeping a strong negative magnetic field in the extracellular space above the nerve cell body allowing the nerve cell body and the intercellular space to remain in a negative polarity resting state (−55 mV) so long as the exemplary device is located on the skin in the area above the nerve cell body. The device reinforces a natural negative repulsion magnetic field keeping the nerve cell body in a resting state and not excitable.

### Method of Use

The present invention provides a method for pain remediation and relief. In one embodiment, the method of the present invention provides easy application of the therapeutic device on different body appendages and curvatures. The method of the present invention may reduce medical costs and help allow individuals to continue activities of daily living while avoiding medication side effects.

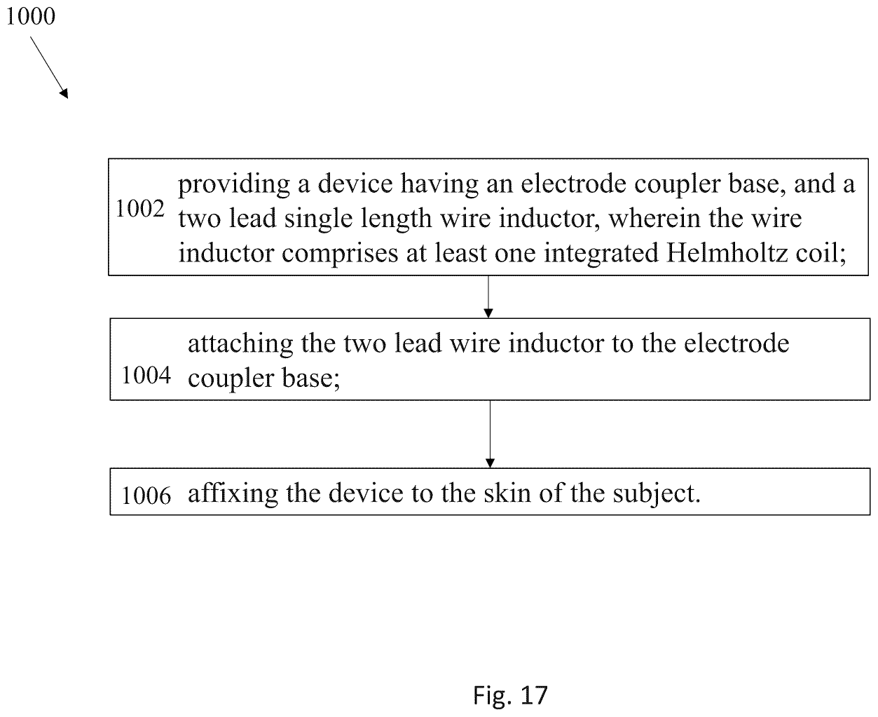

Referring now to FIG. 17, an exemplary method 1000 of using the device is depicted. Method 1000 begins with step 1002, wherein a device having an electrode coupler base, and a two lead single length wire inductor, wherein the wire inductor comprises at least one integrated Helmholtz coil is provided. In step 1004, the two lead wire inductors are attached to the electrode coupler base. In Step 1006, the device is affixed to the skin of the subject. In one embodiment, when assembled, the therapeutic device forms a closed ground loop electromagnetic circuit when affixed to skin surfaces. When affixed to external skin, the device may relieve pain in three ways: first by attracting low value Pauling Units sodium ions in the extracellular region to the exemplary device by using high value electropositive Pauling Unit elements in the device; second by dissipating magnetic fields in the device inductor wire Helmholtz coil; and third by reinforcing a natural negative repulsion of like magnetic fields keeping the nerve cell body in a negative polarity resting state. The combination of concepts suppress a voltage action potential waveform in the nerve cell bodies preventing transmission of pain signals in the area of the exemplary device field of influence.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A therapeutic device for pain relief comprising:
an electrode coupler base, and
a two lead single length wire inductor, wherein the wire inductor comprises at least one integrated Helmholtz coil.

2. The device of claim 1, wherein the electrode coupler base is made from a material selected from the group consisting of: alloys of stainless steel, iron, zinc, nickel chromium, high carbon steel, low carbon steel, silver sheets and combinations thereof.

3. The device of claim 1, wherein the electrode coupler base has a shape selected from the group consisting of: arched, round, rectangle, square, keystone, flat, curve, hollow curve tube, straight tube shape and combinations thereof.

4. The device of claim 1, wherein the electrode coupler base further comprises at least two slot openings, configured to allow attachment of the electrode coupler to the two lead wire inductor.

5. The device of claim 4, wherein the two lead wire inductor are inserted within the at least two slot openings and completing a closed loop electromagnetic circuit, allowing the attraction of sodium ions.

6. The device of claim 1, wherein the at least one integrated Helmholtz coil comprises at least one turn in the coil.

7. The device of claim 1, wherein the at least one integrated Helmholtz coil is positioned anywhere on the length of the two lead wire inductor.

8. The device of claim 1, wherein the two lead wire inductors further comprise a securing clasp positioned at each end of the two lead wire inductors and is configured to secure the electrode coupler base to the two lead wire inductors.

9. The device of claim 8, wherein the securing clasp has a length ranging between approximately 3-5 mm.

10. The device of claim 1, wherein the one lead of the wire inductor further comprises a sharp ninety degree left bend.

11. The device of claim 1, wherein the electrode coupler base is shaped as an arched half round and half rectilinear with two triangular shape cutouts that are used as compression hems.

12. The device of claim 1, wherein the two lead wire inductor is made from a material selected from the group consisting of: alloys of stainless steel, iron, zinc, nickel chromium, high carbon steel, low carbon steel, silver sheets and combinations thereof.

13. The device of claim 1, wherein the electrode base coupler is attached to two lead wire inductor via a method selected from the group consisting of: high strength epoxy adhesives, metal alloy soldering, Bondic [TM] Ultraviolet activated glue, and combinations thereof.

14. The device of claim 1, wherein the device is fixed to the skin of a subject by a method selected from the group consisting of: an adhesive bandage tape, an appendage fabric material, a fabric hook and loop belt material or combinations thereof.

15. A method of providing pain relief to a subject comprising the steps of:
providing a device having an electrode coupler base, and a two lead single length wire inductor, wherein the wire inductor comprises at least one integrated Helmholtz coil;
attaching the two lead wire inductor to the electrode coupler base; and
affixing the device to the skin of the subject.

16. The method of claim 15, wherein the electrode base coupler is attached to two lead wire inductor via a method selected from the group consisting of: high strength epoxy adhesives, metal alloy soldering, Bondic [TM] Ultraviolet activated glue, and combinations thereof.

17. The method of claim 15, wherein the electrode coupler base further comprises at least two slot openings, configured to allow attachment of the electrode coupler to the two lead wire inductor.

18. The method of claim 17, wherein the two lead wire inductor are inserted within the at least two slot openings and completing a closed loop electromagnetic circuit, allowing the attraction of sodium ions.

19. The method of claim 15, wherein the device is fixed to the skin of a subject by a method selected from the group consisting of: an adhesive bandage tape, an appendage fabric material, a fabric hook and loop belt material or combinations thereof.
